(19) United States
(12) Patent Application Publication
(10) Pub. No.: US 2018/0361136 A1
(43) Pub. Date: Dec. 20, 2018

(54) METHOD OF ELECTRO-THERAPEUTIC STIMULATION

(71) Applicant: Paul Robert Matthews, JR., Vernon Hills, IL (US)

(72) Inventor: Paul Robert Matthews, JR., Vernon Hills, IL (US)

(21) Appl. No.: 15/624,695

(22) Filed: Jun. 15, 2017

(51) Int. Cl. A61N 1/04; A61N 1/378

(52) U.S. Cl. CPC A61N 1/0452; A61N 1/3785

(57) ABSTRACT

A method of electro-therapeutic stimulation is disclosed herein. The method includes the steps of attaching electrodes to a practitioner and a patient, physically contacting the patient's body with the practitioner's body, and transmitting electrical current to the patient's body through the practitioner's body. This method of treatment for soft tissue injuries and disorders enables the practitioner to integrate electrical muscle stimulation with any form of soft tissue or manual therapy, stretching, or movement.

### FIELD OF INVENTION

This application is in the field of electro-therapeutic stimulation.

### BACKGROUND

Current methods of treatment of outcomes of soft tissue injuries and disorders typically involve some manner of physical manipulation or massage. Such methods may include manual massage, machine massage, Rolfing, Muscle Activation Techniques (MAT®), as well as Active Release Techniques® (ART®). Usually, in the course of such treatments, when force or pressure is normally applied to an area that is injured, inflamed, or adhesed, it creates a notable reaction of pain or discomfort and a desire for the patient to guard or protect the area and to move away from the point of contact. While this reaction is a natural response to a stimulus of pain or discomfort, it poses difficulty during attempts to treat the soft tissue injury or disorder.

Additionally, the therapeutic techniques of electrical muscle stimulation (EMS) may also be used for the promotion of lymph flow, recovery from injury or fatigue, the treatment of musculoskeletal conditions, and for the purposes of physical therapy and training. EMS is an accepted and commonly used modality by a variety of practitioners including, but not limited to, physiotherapists, physical therapists, chiropractors, athletic trainers, acupuncturists, and osteopaths, as well as by patients themselves. EMS is used for treating pain, stimulating muscles, and helping the body to heal faster and more efficiently from soft tissue injuries. EMS is typically applied through the use of electrodes being placed on a patient's body and connected to an appliance that creates electrical current pulses to be passed on to a patient's body. An alternative treatment consists of the application of a pulsing electric field by a practitioner to a patient without passing current to the patient's body. This treatment is realized by the application of electrodes on both the therapist and the patient and by the electrical isolation of the therapist from the patient through the utilization of a non-conductive material at the point of contact between the therapist and the patient.

Current methods that combine EMS with manual therapy techniques require either the use of a non-conductive gloves, varying types of electrodes attached to or sewn into fabric for self or patient care, or various probe-like tools. Such techniques can produce undesirable or ineffective responses from a patient and do not give the practitioner the ability to accurately feel the area being treated. Moreover, currently available methods do not permit the application of electric current directly to the specific area being palpated by a practitioner.

Accordingly, there's a need for a treatment method that incorporates both EMS, as well as manual physical therapy, in a manner that allows a practitioner to both accurately feel what the practitioner is doing as well as deliver the electrical stimulation precisely to the affected area being treated. These and other features and advantages of the present invention will be explained and will become obvious to one skilled in the art through the summary of the invention that follows.

### SUMMARY

The present invention is directed to methods of electro-therapeutic stimulation. A version of the invention is a method in which a combination of any form of manual or soft tissue therapy is coupled with the use of a powered muscle stimulator. This version of the invention involves grounding one electrode to the patient and connecting another electrode to the practitioner, thereby enabling the practitioner to act as a conductor when the practitioner touches the patient and to transmit electrical current through the practitioner's hands, knuckles, elbows, or any other part of their body. This method of treatment for soft tissue injuries and disorders enables the practitioner to create an unprecedented integration of electrical muscle stimulation with any form of soft tissue or manual therapy, stretching, or movement.

In accordance with a version of the present invention, a method of electro-therapeutic stimulation comprises the steps of: attaching at least one electrode to a practitioner; attaching at least one electrode to a patient; physically contacting said patient's body; and transmitting electrical current to said patient's body through said practitioner's body.

According to an embodiment of the present invention the method of electro-therapeutic stimulation comprises the steps of: attaching at least one electrode of a first set of electrodes to a practitioner; attaching at least one electrode of a second set of electrodes to a patient; generating electrical current; physically contacting said patient's body; and transmitting electrical current from said at least one electrode of a first set of electrodes through said practitioner's body to said patient's body.

According to an embodiment of the present invention the step of generating electrical current comprises the operation of a current generating apparatus and in another embodiment thereof the step of attaching at least one electrode of a second set of electrodes to a patient further comprises the step of grounding said at least one electrode of a second set of electrodes.

According to an embodiment of the present invention the step of physically contacting said patient's body comprises the step of applying a therapeutic technique which may be palpation, massage, Rolfing, muscle activation technique, active release technique, or stretching.

According to an embodiment of the present invention the current generating device is an electrical muscle stimulation device and in another embodiment the step of physically contacting said patient's body comprises the step of touching said patient with said practitioner's body part selected from the group of hand, knuckle, elbow, and arm.

According to an embodiment of the present invention a method of electro-therapeutic stimulation comprises the steps of: providing an electrical muscle stimulation device; attaching at least one electrode of a first set of electrodes of said electrical muscle stimulation device to a practitioner; attaching at least one electrode of a second set of electrodes of said electrical muscle stimulation device to a patient; generating electrical current by operation of said electrical muscle stimulation device; and transmitting electrical current from said at least one electrode of a first set of electrodes through said practitioner's body to said patient's body.

### BRIEF DESCRIPTION OF THE DRAWINGS

These and other features, aspects, and advantages of the present invention will become better understood with regard to the following description, appended claims, and accompanying drawings where:

FIG. 1 shows a process flow of performing the inventive method according to a version of the present invention;

FIG. 2 shows a block diagram of alternative ways of generating current according to a version of the present invention; and

FIG. 3 shows an alternative ways of transmitting current to a patient according to a version of the present invention.

### DETAILED DESCRIPTION

In the Summary above, in the Detailed Specification, the Claims below, and in the accompanying drawings, reference is made to particular features including method steps of the invention. It is to be understood that the disclosure of the invention in this specification includes all possible combinations of such particular features. For example, where a particular feature is disclosed in the context of a claim, that feature can also be used to the extent possible, in combination with and/or in the context of other particular aspects and embodiments of the invention and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e. contain only) components A, B, and C or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context exclude that possibility).

This invention is described in the following description with reference to the Figures, in which like reference numbers represent the same or similar elements. While this invention is described in terms of modes for achieving this invention's objectives, it will be appreciated by those skilled in the art that variations may be accomplished in view of these teachings without deviating from the spirit or scope of the present invention. The embodiments and variations of the invention described herein, and/or shown in the drawings, are presented by way of example only and are not limiting as to the scope of the invention.

Unless otherwise specifically stated, individual steps and elements of the invention may be omitted or modified, or may have substituted therefore known equivalents, or as yet unknown substitutes such as may be developed in the future or such as may be found to be acceptable substitutes in the future. The invention may also be modified for a variety of applications while remaining within the spirit and scope of the claimed invention, since the range of potential applications is great, and since it is intended that the present invention be adaptable to many such variations.

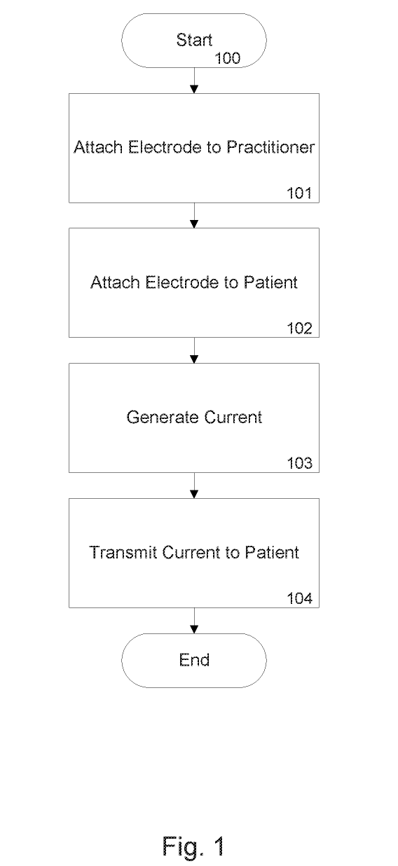

Versions of the present invention generally relate to methods of electro-therapeutic stimulation. More specifically, as illustrated in FIG. 1, a preferred version of the present invention 100 comprises attaching at least one electrode of a first set of electrodes to a practitioner 101; attaching at least one electrode of a second set of electrodes to a patient 102; generating electrical current 103; physically contacting said patient's body and thereby transmitting electrical current from said at least one electrode of a first set of electrodes through said practitioner's body to said patient's body 104.

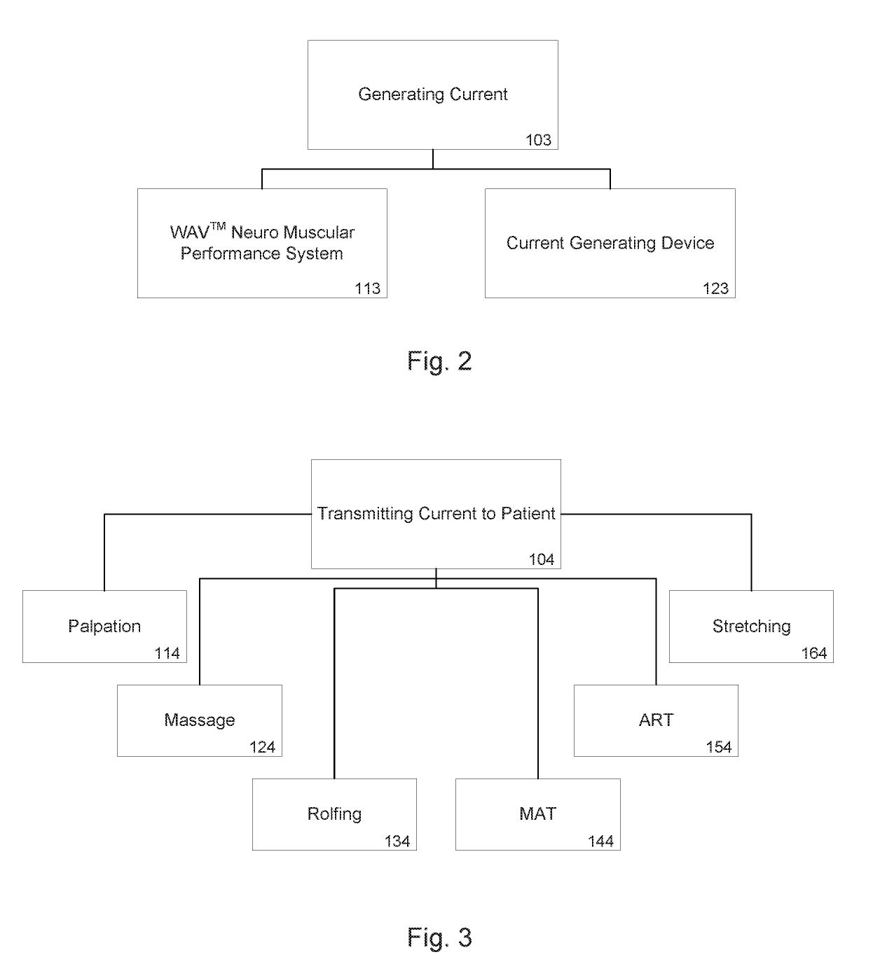

In accordance with a version of the present invention the current may be generated 103 through the use of a current-generating EMS apparatus including, but not limited to, the operation of the WAV™ Neuro Muscular Performance System 113 as indicated in FIG. 2. Alternative current generating EMS devices 123 may be used in the implementation of the present invention but may not be as effective and may create different sensations in the patient from those created by the WAV™ Neuro Muscular Performance System.

A preferred version of the present invention is intended in part to significantly improve the treatment outcomes of soft tissue injuries and disorders by allowing a practitioner the ability to use their own body as the tool for which they conduct the electrical current into the patient 104. Accordingly, the preferred version of the present invention permits the practitioner to obtain pinpoint palpation of the structures one is contacting on the patient; enhanced depth and pressure control with increased sensory and biofeedback compared to the use of an apparatus, probe or tool; and, as indicated in FIG. 3, the ability to combine EMS with any soft tissue technique including, but not limited to palpation 114, massage 124, rolfing 134, MAT 144, ART 154, and stretching 164. As should be understood, although the various embodiments of the present invention could be incorporated into almost all soft tissue treatments, it is particularly effective in treatments where the end goal is the break-up of scar tissue, adhesions, fibrotic tissue, or trigger points; lengthening shortened tissue; improving blood flow into restricted tissue; releasing hypertonic tissue; reducing swelling and inflammation of affected tissue; releasing myofascial restriction to improve mobility and flexibility; improving delayed onset of muscle soreness; and general improvement of the range of motion.

Most important, however, is the outcome and reaction of a patient's body when EMS is integrated with a soft tissue therapeutic touch technique such as those mentioned above. While typically, when pressure is applied to a treatment area that is injured, inflamed, or adhesed, due to pain or discomfort, the patient has a natural tendency to either guard the area or to pull away from the point of contact. However, in accordance with the preferred version of the present invention, when EMS is combined with manual or soft tissue therapy this natural reaction is overcome. This is because such a combination has a calming effect on the nervous system when applied by a practitioner working on such areas and produces a significantly less painful reaction due to the direct application of electric current to the affected area. Furthermore, the method of the preferred version of the present invention speeds up the healing process by helping to flush new blood into the affected area and further breaks up adhesions, as the electrical current helps override the central nervous systems fight or flight response while performing soft tissue therapy making it easier to apply pressure into painful tissues.

Furthermore, the method in accordance with a version of the present invention incorporates a process which contracts the actual muscle and surrounding tissue one is trying to manipulate thereby creating highly unique working conditions for any form of manual or soft tissue therapy. Accordingly, a practitioner is thereby able to create more of a resistance to work against through the contraction of muscle and adjacent tissues as the practitioner applies pressure which, in turn, significantly increases the quality and effectiveness of soft tissue and manual therapy work. The principal underlying premise here is that tissue work is required to break up scar tissue, release tight muscles, and remove restrictions impeding joint mobility. The combination of EMS with manual or soft tissue therapeutic techniques has a whole host of additional benefits including facilitation of muscle reeducation, improving blood flow, and the facilitation of the breakup of scar tissue. These effects are especially profound and significant when integrated with movement. The preferred version of the present invention elicits a variety of capabilities, as well as a variety of potential therapy protocols to be followed by a skilled practitioner.

While previous attempts to combine EMS with manual therapy techniques required the use of non-conductive gloves, the use of varying types of electrodes sown into fabric, or use various probe like tools, these attempts are ineffective in soliciting the desired response and do not give the practitioner the ability to actually feel what the practitioner is doing. Further, the former attempts did not transmit the current through the practitioner's body to the patient. Consequently, the versions of the present invention permit a more precise and effective application for a wide range of purposes. Since muscles and tissues in our bodies have depth, the versions of the present invention enable a practitioner to access deeper structures with more precision compared to the typical methods of applying electrodes to the body. Furthermore, because the various versions of the present invention all involve the use of the practitioners body in order to conduct current to the patient, as opposed to an external tool or apparatus, the methods described herein give the practitioner the ability to more accurately sense and feel what they are actually doing and more precisely treat the afflicted area. This result cannot be achieved if the current is being transferred through the use of another external tool or apparatus, as it is impossible to simultaneously feel what the practitioner is manipulating or working on through such object with the same effectiveness as with one's hands, wherein the current is being transferred directly to the point of contact. While versions of the present invention envision the transmission of the electric current from the practitioner's body through direct unimpeded contact with the patient's body, alternative variations of the present invention may incorporate the use of a conductive wearable element including, but not limited to, a conductive glove, for the transmission of current from the practitioner's body to the patient's body. It should be understood that incorporation of elements capable of being worn on any part of a practitioner's body that comes in contact with a patient during treatment such as, but not limited to, the fingertips, hands, elbows, and knuckles is also envisioned in accordance with the various embodiments of the present invention. The purpose of incorporating such a wearable element may be manifold and may include among other things: increasing the sensation felt by a patient during treatment while simultaneously decreasing sensation felt by the practitioner; creating a more conductive medium between practitioner and patient for the conduction of electric current to the latter; and allowing the practitioner to practice the various embodiments of the present invention without actually having to come into direct skin-to-skin contact with the patient.

Additionally, the preferred versions of the present invention provide very powerful effects on the patient's nervous system, as they help to override the aforementioned unwanted protective mechanisms. Furthermore, the methods are useful for breaking down compensational patterns and helping to reeducate the muscles to optimal function, wherein the muscles are manipulated or released back to a more balanced or neutral position. The effectiveness of the method according to the versions of the present invention arise from the understanding that that chronic musculoskeletal pain in the human body is rooted in two basic components: (1) areas where the body has been overworked and/or where overcompensation results in pain, tightness, poor mobility, adhesions, and poor stability; and (2) areas where the muscles have become weakened and have ultimately come to be avoided by the brain's neurological pathways. Consequently, the human body will ultimately default to the path of least resistance, even if it causes a person to continually overuse and abuse the same muscles and tissues to the point that such person develops degenerative joint and tissue damage. Accordingly, to address these underlying causes, the versions of the present invention are able to simultaneously break down the soft tissue restrictions by sending electrical current into the specific compromised tissue areas to help those tissues heal more efficiently and return to optimal function. When the methods described herein are performed according to specific protocols by a skilled and trained practitioner, one can neutralize a soft tissue restriction and then subsequently retrain the neuromuscular system to return to proper function. Essentially, through the application of the preferred version of the present invention, the methods described herein help break down what impedes the body from moving well and also helps stimulate and activate the weakened and damaged muscle and adjoining tissues.

The benefits of the various embodiments of the present inventions are manifold. Usually, a patient may be aware of the general area where discomfort or pain is located, but may be unaware of the specific locations of where the pain begins and ends as those locations may not be in the same place. This often causes difficulty because trigger points may refer pain into an area that is completely different from that where the cause of the pain or the tissue dysfunction may be located. Accordingly, by implementing any of the various embodiments of the present invention, as a practitioner comes in contact with the patient's body while performing a therapeutic technique, a patient would experience an enhanced sensation compared to that of typical soft tissue techniques. Depending on the nature of a patient's injury or dysfunction, the enhanced sensation that a patient may feel may, for example, be a deeper ache, pain, or discomfort as in the case of weakened or damaged tissue, or a more pleasant relieving sensation as in the case of hypertonic or overactive tissues. The intensity of these sensations may be controlled by the output level of the current generating apparatus and the technique being implemented in accordance with an embodiment of the present invention. Accordingly, such enhanced sensation and feedback provides the practitioner additional insight and ability to better identify the sources and causes of the dysfunction and/or pain and to better address them in an appropriate and targeted manner. This benefit may be better understood through the following example. If a patient comes to a practitioner with complaints of right side lumbar pain, a practitioner employing the preferred embodiment of the present invention would be able to determine that the primary source of the issue is in fact the psoas muscle by identifying that pressure on the tight origin attachment of the psoas muscle and restricted tissue refers pain along the entire path of compensation including the lower back, core, and hip flexor. Thus, the method according the preferred embodiment of the present invention facilitates a more precise identification of the causes of pain or undesirable, highlighting the muscles and tissues involved therein and aids in the targeted treatment thereof.

The method, in accordance with a version of the present invention, has the ability to revolutionize the rehabilitation, recovery, and performance industries, as well as most uses of an electro therapeutic device. The various versions of the present invention may be used in conjunction with existing treatment platforms or on their own following protocols performed by skilled practitioners. It should be understood by one skilled in the art that, for each region of the body, specific protocols exist that the practitioner or end user may follow or combine with an endless array of other therapies.

In view of the foregoing, it will now be appreciated that elements of the block diagrams and flowchart illustrations support combinations of means for performing the specified functions, combinations of steps for performing the specified functions, and so on.

Although the present innovation has been described in considerable detail with reference to certain preferred versions thereof, other versions are possible. For example a variety of other soft tissue manipulation and therapeutic techniques may be incorporated within the versions of the present invention. Similarly, the present invention is envisioned to operate through the use of wearable conductive elements or apparatuses on the practitioner's body which permit physical manipulation of the affected area simultaneously with the transmission of electrical current directly thereto. Therefore, the spirit and scope of the appended claims should not be limited to the description of the preferred versions described herein.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

1. A method of electro-therapeutic stimulation, the method comprising the steps of:
attaching at least one electrode of a first set of electrodes to a practitioner;
attaching at least one electrode of a second set of electrodes to a patient;
generating electrical current;
physically contacting said patient's body; and
transmitting electrical current from said at least one electrode of a first set of electrodes through said practitioner's body to said patient's body.

2. The method of electro-therapeutic stimulation of claim 1 wherein the step of generating electrical current comprises the operation of a current generating apparatus.

3. The method of electro-therapeutic stimulation of claim 1 wherein the step of attaching at least one electrode of a second set of electrodes to a patient further comprises the step of grounding said at least one electrode of a second set of electrodes.

4. The method of electro-therapeutic stimulation of claim 1 wherein the step of physically contacting said patient's body comprises the step of applying a therapeutic technique.

5. The method of electro-therapeutic stimulation of claim 2 wherein said therapeutic technique is palpation.

6. The method of electro-therapeutic stimulation of claim 2 wherein said therapeutic technique is massage.

7. The method of electro-therapeutic stimulation of claim 2 wherein said therapeutic technique is Rolfing.

8. The method of electro-therapeutic stimulation of claim 2 wherein said therapeutic technique is a muscle activation technique.

9. The method of electro-therapeutic stimulation of claim 2 wherein said therapeutic technique is active release technique.

10. The method of electro-therapeutic stimulation of claim 2 wherein said therapeutic technique is stretching.

11. The method of electro-therapeutic stimulation of claim 2 wherein said current generating apparatus is an electrical muscle stimulation device.

12. The method of electro-therapeutic stimulation of claim 1 wherein the step of physically contacting said patient's body comprises the step of touching said patient with said practitioner's body part selected from the group of hand, knuckle, elbow, and arm.

13. The method of electro-therapeutic stimulation of claim 2 wherein the step of physically contacting said patient's body comprises the step of touching said patient with said practitioner's body part selected from the group of hand, knuckle, elbow, and arm.

14. A method of electro-therapeutic stimulation, the method comprising the steps of:
providing an electrical muscle stimulation device;
attaching at least one electrode of a first set of electrodes of said electrical muscle stimulation device to a practitioner;
attaching at least one electrode of a second set of electrodes of said electrical muscle stimulation device to a patient;
generating electrical current by operation of said electrical muscle stimulation device; and
transmitting electrical current from said at least one electrode of a first set of electrodes through said practitioner's body to said patient's body.

15. The method of electro-therapeutic stimulation of claim 14 wherein the step of transmitting electrical current comprises performing a therapeutic technique on said patient.

16. The method of electro-therapeutic stimulation of claim 15 wherein said therapeutic technique is one selected from the group comprising palpation, massage, rolfing, muscle activation technique, active release technique, and stretching.

17. The method of electro-therapeutic stimulation of claim 14 wherein the step of transmitting electrical current comprises the step of touching said patient with said practitioner's body part selected from the group of hand, knuckle, elbow, and arm.
